(19) United States
(12) Patent Application Publication
(10) Pub. No.: US 2010/0086495 A1
(43) Pub. Date: Apr. 8, 2010

(54) COMPOSITIONS AND METHODS FOR ENHANCING TRANSMUCOSAL DELIVERY

(75) Inventor: Yosef Rubinstein, Ness Ziona (IL)

Correspondence Address: ROTHWELL, FIGG, ERNST & MANBECK, P.C., 1425 K STREET, N.W., SUITE 800, WASHINGTON, DC, 20005, US

(73) Assignee: DERMA-YOUNG LTD., Ness Ziona (IL)

(21) Appl. No.: 12/527,299

(22) PCT Filed: Feb. 14, 2008
(86) PCT No.: PCT/IL08/00192

§ 371 Date: Dec. 14, 2009

Related U.S. Application Data
(60) Provisional application No. 60/901,345, filed on Feb. 15, 2007.

(51) Int. Cl. A61K 9/68; A61K 31/10; A61K 31/167; A61K 31/415; A61K 31/4045; A61K 31/135; A61K 31/137; A61K 31/4525; A61K 36/00

(52) U.S. Cl. 424/48; 514/711; 514/629; 514/404; 514/415; 514/646; 514/649; 514/321; 424/725; 514/658

(57) ABSTRACT

The present invention provides transmucosal pharmaceutical or nutraceutical compositions and methods for enhancing transmucosal delivery of pharmaceutical and nutraceutical ingredients through use of methylsulfonylmethane (MSM) as a transmucosal delivery enhancer. In particular, the invention provides transmucosal compositions comprising an active agent selected from a non-steroid anti-inflammatory drug (NSAID); an analgesic; a migraine medication; a menopause medication; a sleep disorder medication; an erectile dysfunction medication, an appetite suppressant, a vitamin, a food supplement and a macromolecule. In certain particular exemplary formulations the appetite suppressant is DL-phenylalanine, and the vitamin is B12.

### FIELD OF THE INVENTION

The present invention relates to compositions and methods for transmucosal delivery of pharmaceutical agents, including macromolecular compounds, and nutraceutical ingredients. The invention involves use of methylsulfonylmethane as a transmucosal delivery enhancer.

### BACKGROUND OF THE INVENTION

### Drug Delivery Via Oral Mucosa

Medicaments taken by mouth and swallowed are absorbed first into the blood perfusing the gastrointestinal (GI) tract. The venous drainage from the GI tract is first passed into the blood perfusing the liver. This means that medicaments absorbed from the lumen of the gastrointestinal tract are immediately presented to the liver, the major detoxifying organ of the body. In addition to protecting the organism from ingested toxins, the liver also metabolizes medicaments, which may be inactivated by first pass metabolism in the liver. Blood from the liver then returns to the left side of the heart via the hepatic portal vein and reaches the rest of the systemic circulation. This first pass through the liver may result in the removal of a substantial proportion of an ingested medicament.

Accordingly, other routes of drug administration have been investigated, including those involving transport across the mucous membranes. Of the various mucous membranes (e.g., oral, rectal, vaginal, ocular, nasal), drug delivery via the mucous membranes in the oral cavity seems to be the most easily tolerated by patients. In addition to avoiding the problems with traditional oral administration, drug delivery via the mucous membranes of the oral cavity has certain other advantages, due to the properties of the oral mucosa itself. For example, the mucous membranes of the oral cavity are highly vascularized and well supplied with lymphatic drainage sites.

In general, the mucous membranes of the oral cavity can be divided into five main regions: the floor of the mouth (sublingual), the cheeks (buccal), the gums (gingival), the roof of the mouth (palatal), and the lining of the lips. These regions differ from each other with respect to their anatomy, drug permeability, and physiological response to drugs. For example, in terms of relative permeability, the sublingual region is more permeable than the buccal region, which is more permeable than the palatal region. This permeability is generally related to the relative thickness and degree of keratinization of these membranes, with the sublingual mucosa being relatively thin and non-keratinized, the buccal mucosa being thicker and non-keratinized, and the palatal mucosa being intermediate in thickness, but keratinized.

In addition to the differences in permeability of the various mucous membranes, the extent of drug delivery is also affected by the properties of the drug to be delivered. The ability of a molecule to pass through any mucous membrane is dependent upon its size, its lipid solubility, and the extent to which it is ionized, among other factors.

Certain areas of the alimentary canal have a venous drainage, which does not involve a first pass through the liver. These areas (the mucous membrane of the buccal cavity, under the tongue and the nasopharynx) drain directly into the left side of the heart. The avoidance of the first pass effect is the rationale for the use of buccal and sublingual formulations. The delivery through buccal mucosa allows a lower amount of the composition to be effective immediately.

However, because the continuous secretion of saliva rapidly washes dissolved drugs out of the oral cavity, sublingual and buccal administration of drugs have been most useful for drugs, such as nitroglycerin, which are very rapidly absorbed through the oral mucosa. In order to keep a drug in contact with the oral mucosa for a longer period of time, sustained release dosage forms especially adapted for transmucosal administration of drugs have been developed. These have generally comprised a drug dispersed in a matrix, which slowly releases the drug by diffusion from the matrix or by slow dissolution or erosion of the matrix. In order to retain the dosage form within the mouth, it may be bonded to an adhesive patch or the dosage form itself may be provided with an adhesive layer, which adheres to the mucosa. Alternatively, the dosage form itself may adhere to the mucosa and slowly dissolve, releasing the drug contained therein.

U.S. Pat. No. 4,572,832 discloses a soft buccal dosage form containing (1) a medicament to be absorbed through the oral mucosa, (2) a water-soluble protein, (3) a polyhydric alcohol, and (4) a fatty acid ester or/and a carboxyvinyl polymer, which can be used for administration to the mucous membrane of the mouth.

U.S. Pat. No. 4,764,378 discloses buccal dosage forms for transmucosal administration of drugs comprising a pharmaceutical compound dispersed in an erodible matrix comprising from about 20 to about 75 percent by weight of a low molecular weight polyethylene glycol component, from about 2 to about 65 percent by weight of a medium or high molecular weight polyethylene glycol component, from about 1% to about 40% percent by weight of an auxiliary high molecular weight polymer.

U.S. Pat. No. 5,346,701 discloses a system for mucosal administration of a macromolecular drug, comprising an inner drug/enhancer/polymer layer having one surface adapted to contact the mucosal tissue of the oral cavity and adhere thereto, said inner layer containing a bile salt enhancer, a hydrophilic polymer and a macromolecular drug having a molecular weight of at least 500 daltons inter alia calcitonin or heparin.

U.S. Pat. No. 4,713,243 to Schiraldi et al. discloses a thin film capable of adhering to a wet mucous surface which comprises a bioadhesive layer consisting of hydroxypropyl cellulose, polyethylene oxide, a plasticizer, a medicament, and optionally a water-insoluble polymer. The film disclosed in U.S. Pat. No. 4,713,243 is useful for controlled release of a medicament such as anesthetics, anti-inflammatories, antihistamines, antibiotics, and antibacterials.

U.S. Pat. Nos. 5,948,430, 6,177,096, 6,284,264, 6,592,887, and 6,709,671 to Zerbe et al. disclose mucoadhesive films capable of rapidly dissolving and adhering to the oral cavity comprising a water-soluble polymer and a pharmaceutically or cosmetically active ingredient and methods of use thereof.

### Vitamin B12

Vitamin B12 (also known as cobalbumin) deficiency is caused by pernicious anemia, food-cobalbumin malabsorption, vegetarianism and other deficiency states, and has a reported prevalence of up to 29%. The condition may be corrected by intramuscular injection of vitamin B12, a method which has the disadvantages of being painful for patients, and difficult to administer. Alternately, it may be treated by oral administration of vitamin B12; however this method is associated with the disadvantage of requiring large doses (at least 500-1000 μg) in order to supply the daily requirement of 1 to 2.5 μg, due to the poor rate of absorption in the gastrointestinal tract. Sublingual dosage forms of vitamin B12 are also commercially available, and while it has been purported that such dosage forms provide enhanced bioavailability, treatment of vitamin B 12 deficient subjects with conventional oral vitamin B12 or with sublingual vitamin B 12 has been shown to result in the same extent of increase in serum concentration levels of the vitamin (Sharabi et al (2003) Br J Clin Pharmacol 56: 635-638).

Accordingly, there remains an unmet need for dosage forms of vitamin B12 which provide increased bioavailability over prior art formulations.

### Methylsulfonylmethane

Methylsulfonylmethane (MSM), also known as dimethyl sulfone or organic sulfur, is a naturally occurring sulfur-containing compound found in a variety of fruits, vegetables, grains, mammal's milk and animals including humans. MSM is the primary oxidative metabolite product of dimethyl sulfoxide in humans.

The toxicity of methylsulfonylmethane was evaluated in rats at a dose five to seven times the maximum recommended dose in humans is (Horvath et al., 2002, Food Chem. Toxicol. 40(10):1459-1462). MSM is well tolerated in rats at an acute dose of 2 g/kg and at a subacute chronic dose of 1.5 g/kg.

MSM is sold as a dietary supplement that is marketed with a variety of claims and is commonly used (often in combination with glucosamine and/or chondroitin) for treatment or prevention of osteoarthritis. Kim et al (2006) disclose a pilot study which indicates that MSM has potential for the relief of osteoarthritis of the knee (Kim et al (2006) Osteoarthritis Cartilage 14(3):286-94).

Published patents relating to the use of methylsulfonylmethane to prevent, treat or relieve various conditions include: U.S. Pat. Nos. 6,440,391 and 5,569,679 (management of snoring); U.S. Pat. No. 4,973,605 (relief of pain and nocturnal cramps and reduction of stress-induced death in animals); U.S. Pat. No. 4,559,329 (reduction of gastric upset and allergic reactions); and U.S. Pat. No. 4,447,469 (as a topical preparation to soften skin, nails and other tissues).

U.S. Pat. No. 4,568,547 discloses use of methylsulfonylmethane as a tableting and granulating excipient for pharmaceutical ingredients which are unstable in the presence of moisture. According to the disclosure, the invention relates to solid pharmaceutical compositions adapted for oral ingestion in the form of tablets, granules or a free-flowing powder, consisting essentially of an intimate physical mixture of an active agent which is storage unstable in the presence of moisture, and a carrier which consists essentially (i.e. at least 95%) of methylsulfonylmethane, wherein the mixture is essentially anhydrous.

U.S. Patent Application Publication No. 2005/0181048 discloses compositions and methods for producing timed or retarded release neutraceutical formulations, inter alia those containing methylsulfonylmethane as the active substance. According to the disclosure, pellets for timed or retarded release of MSM comprise MSM in an amount of about 60 to 95% by weight, a saccharide in an amount of about 1.5 to 15% by weight, as well as additional components inter alia a plasticizer, an agglutinative and a lubricant in specified percentages.

U.S. Pat. No. 6,444,234 discloses a liquid carrier composition effective for the transdermal delivery of a medicament having a given polarity, said formulation comprising (a) at least one non-aqueous non-toxic solvent; (b) limonene, lemon oil or mixture of limonene and lemon oil; (c) methylsulfonylmethane; (d) a skin stabilizer (e) a solute modifier; and (f) adenosine triphosphate (ATP) or a compound which induces generation of cyclic adenosine 3′5′monophosphate cAMP in situ or cyclic guanosine monophosphate (cGMP) in situ.

U.S. Pat. No. 6,416,772 discloses a liquid composition applied transdermally for relief of pain comprising alcohol in an amount by weight of about 57 to about 91 percent; glycerin in an amount by weight of about 1 to about 12 percent; an analgesic agent in an amount by weight of about 2 to about 28 percent, the analgesic agent comprising a derivative of salicylic acid; methylsulfonylmethane in an amount by weight of about 0.02 to 5 percent; and emu oil in an amount by weight of about 0.01 to 3 percent, the liquid composition permeating skin to relieve pain.

However, none of the background art discloses or suggests that methylsulfonylmethane enhances transmucosal delivery.

### Obesity and Appetite Suppression

Obesity is a chronic, essentially intractable metabolic disorder of ever-increasing prevalence for which no effective treatment is currently known. The obesity rate worldwide is increasing and is currently considered as the core epidemic of the western world in the 21^{st} century. More than 50% of the U.S. population is considered overweight, with >25% diagnosed as clinically obese. Statistical data show that obesity starts at an increasingly younger age; 15% of children and juveniles suffer from overweight, a rate which is 3 fold higher than that reported 25 years ago.

Obesity is a recognized risk factor for diabetes mellitus type 2, cardiovascular disease, hypertension, atherosclerosis, congestive heart failure, stroke, gallbladder disease, osteoarthritis, sleep apnea, reproductive disorders such as polycystic ovarian syndrome, cancers of the breast, prostate, and colon, and increased incidence of complications of general anesthesia (Reisin and Alpert, 2005, Am J Med. Sci. 330, 6, 269-272). It reduces life span and carries a serious risk of co-morbidities as described above, as well as complications including infections, varicose veins, acanthosis nigricans, eczema, exercise intolerance, insulin resistance, hypertension hypercholesterolemia, cholelithiasis, orthopedic injury, and thromboembolic disease (Rissanen et al., BMJ 301: 835-7, 1990). Obesity is also a risk factor for the group of conditions called insulin resistance syndrome, or “Syndrome X”.

Therefore, there is a clear economic and medical rationale to develop therapeutic strategies for prevention and control of obesity.

The failure to control food intake in a normal manner has long been thought to be a major cause of obesity. Hunger cravings and extreme appetite urges are part of the cycle of overeating in obese and morbidly obese subjects. A lack of understanding of the control mechanisms by which food intake is regulated in the normal animal or human has, however, prevented an effective clinical application of this concept.

Mechanisms previously postulated to be primarily responsible for physiological appetite control include the increase in blood glucose levels and/or gastric distension resulting from food intake. Subsequent theory holds that these controls are of secondary importance, while the primary mechanism for eliciting satiety and inhibiting feeding involves the release of peptide hormones in the small intestine on preabsorptive contact of food stimuli with mucosal gut receptors. While a number of peptide hormones may participate, the “satiety effect” (appetite suppression) of cholecystokinin (CCK) appears particularly significant (G. P. Smith, Biochemical Pharmacology of Obesity, pp. 407-419, Elsevier Science Publishers, 1983). Clinical applications for controlling appetite include inducement of endogenous release of gut peptides such as CCK.

DL-phenylalanine (DLPA) is an essential amino acid that is a neurotransmitter. Some of DLPA's main functions are hunger reduction, improvements in memory and mental alertness, and depression alleviation. DLPA also has the ability to convert into most of the other 14 vital amino acids necessary to form proteins required from translation of cellular DNA.

The administration of a chemical stimulus to effect the release of the hormone(s) of interest has been proposed, including the use of L-phenylalanine as a pre-absorptive releaser of CCK. U.S. Pat. No. 4,833,128 discloses a dietary supplement formulated to suppress appetite, comprising L-phenylalanine in combination with restricted quantities of protein, carbohydrate, fat and dietary fiber.

U.S. Pat. No. 5,925,377 discloses a dietary supplement composition combining amino acids, minerals, vitamins, herbs, and essential nutrients along with gentle diuretics and digestive enzymes. According to the disclosure, DL-phenylalanine is combined with tyrosine to act as an appetite depressant.

U.S. Patent Application Publication No. 2005/0002927 discloses pharmaceutical compositions and methods comprising at least one Y2 receptor-binding peptide, such as peptide YY (PYY), Neuropeptide Y (NPY) or Pancreatic Peptide (PP) and one or more mucosal delivery-enhancing agents for enhanced nasal mucosal delivery of the peptide YY, for treating a variety of diseases and conditions in mammalian subjects, including obesity.

There is an unmet need for enhanced transmucosal formulations, allowing convenient rapid administration of a pharmaceutical or a nutraceutical via the oral mucosa. In particular there is an unmet need for effective rapid delivery of analgesics, anti-inflammatory drugs, medications for erectile dysfunction, for migraine or for symptoms of menopause, and for improving the delivery of vitamins and agents to be used for suppressing appetite and for the treatment of obesity or an eating disorder.

### SUMMARY OF THE INVENTION

The present invention provides transmucosal pharmaceutical compositions and methods for enhancing transmucosal delivery of pharmaceutical ingredients, including non-steroidal anti-inflammatory drugs, analgesics, migraine medications, menopause medications, sleep disorder medications, erectile dysfunction medications, appetite suppressants and macromolecules. The present invention further provides transmucosal nutraceutical compositions and methods for enhancing transmucosal delivery of nutraceutical ingredients, including vitamins, food supplements and certain appetite suppressants. Advantageously, transmucosal delivery using the compositions and methods of the present invention provides a rapid uptake and response to the agent administered.

The present invention for the first time discloses the finding that methylsulfonylmethane (MSM) enhances transmucosal-oral delivery of a large spectrum of pharmaceutical and nutraceutical ingredients. According to various embodiments, the pharmaceutical ingredient is selected from the group consisting of a non-steroid anti-inflammatory drug (NSAID); an analgesic; a migraine medication; a menopause medication; a sleep disorder medication; an erectile dysfunction medication, an appetite suppressant and a macromolecule. In other embodiments the nutraceutical may be selected from a vitamin, a food supplement and an appetite suppressant. In an exemplary embodiment the appetite suppressant is DL-phenylalanine. Advantageously, transmucosal delivery of DL-phenylalanine enables a rapid response to hunger cravings and appetite urges, which are part of the cycle of overeating in obese and morbidly obese subjects.

According to a first aspect, the present invention provides a pharmaceutical composition for enhanced delivery across the oral mucosa, the composition comprising a pharmaceutical ingredient and methylsulfonylmethane; in a pharmaceutically acceptable carrier, suitable for administration to the oral mucosa. In certain embodiments the carrier comprises an edible oil; purified water and lecithin.

In general, the methylsulfonylmethane is present in the minimal amount suitable to serve as a penetration enhancer and normally does not exceed 30% (w/w) of the total weight of the composition. According to certain embodiments the methylsulfonylmethane does not exceed 25%; preferably it does not exceed 20%; more preferably it does not exceed 10% of the total weight of the composition.

According to one embodiment, the edible oil is a vegetable oil. According to some embodiments, the vegetable oil is selected from the group consisting of cottonseed oil, peanut oil, poppyseed oil, safflower oil, sesame oil, soybean oil, corn oil, olive oil, canola oil and combinations thereof.

According to other embodiments, the composition further comprises at least one excipient selected from the group consisting of a sweetening agent, a flavoring agent, a protecting agent, an antioxidant, a plasticizer, a wax, an elastomeric solvent, a filler material, a preservative, a lubricating agent, a wetting agent, an emulsifying agent, solubilizing agent, a suspending agent, a coloring agent, a disintegrating agent, and combinations thereof.

According to some embodiments, the composition comprises at least one excipient selected from the group consisting of cocoa butter, sucralose, a calcium salt and combinations thereof. According to one embodiment, the calcium salt is calcium carbonate.

According to some embodiments, the composition comprises:

a pharmaceutical ingredient in an amount of about 0.1 to 15%;

methylsulfonylmethane in an amount of about 1 to 30%;

vegetable oil in an amount of about 5% to 40%;

purified water in an amount of about 5 to 30%;

cocoa butter in an amount of about 5 to 40%;

sucralose in an amount suitable to serve as a sweetener;

a calcium salt in an amount of about 1 to 20%, and

lecithin in an amount of about 1 to 20%; wherein the percentages are weight percent based on the total weight of the composition and the total weight of the composition equals 100%.

According to one embodiment, the pharmaceutical ingredient is in an amount of about 1% to 5%;

the methylsulfonylmethane is in an amount of about 1% to 10%;

the vegetable oil is in an amount of about 20% to 40%;

the purified water is in an amount of about 10% to 30%;

the cocoa butter is in an amount of about 20% to 40%;

the sucralose is in an amount of about 0.05% to 0.2%;

the calcium salt is in an amount of about 1% to 20%, and

the lecithin is in an amount of about 1% to 20%.

According to a further embodiment, the pharmaceutical ingredient is in an amount of about 2% to 4%;

the methylsulfonylmethane is in an amount of about 2% to 5%;

the vegetable oil is in an amount of about 25% to 35%;

the purified water is in an amount of about 15% to 25%;

the cocoa butter is in an amount of about 25% to 35%;

the sucralose is in an amount of about 0.07% to 0.15%;

the calcium salt is in an amount of about 5% to 15%, and

the lecithin is in an amount of about 5% to 15%.

According to some embodiments the pharmaceutical ingredient is a non-steroid anti-inflammatory drug (NSAID). According to some embodiments the pharmaceutical ingredient is an analgesic. According to some embodiments the pharmaceutical ingredient is a migraine medication. According to some embodiments the pharmaceutical ingredient is a menopause medication. According to some embodiments the pharmaceutical ingredient is a sleep disorder medication. According to some embodiments the pharmaceutical ingredient is an erectile dysfunction medication. According to some embodiments the pharmaceutical ingredient is an appetite suppressant. According to some embodiments the pharmaceutical ingredient is a macromolecule.

According to one embodiment, the NSAID is selected from the group consisting of ibuprofen (2-(isobutylphenyl)-propionic acid); methotrexate (N-[4-(2,4 diamino 6-pteridinyl-methyl]methylamino]benzoyl)-L-glutamic acid); aspirin (acetylsalicylic acid); salicylic acid; diphenhydramine (2-(diphenylmethoxy)-N,N-dimethylethylamine hydrochloride); naproxen (2-naphthaleneacetic acid, 6-methoxy-9-methyl-, sodium salt, (−)); phenylbutazone (4-butyl-1,2-diphenyl-3,5-pyrazolidinedione); sulindac (2)-5-fluoro-2-methyl-1-[[p-(methylsulfinyl)phenyl]methylene-]-1-H-indene-3-acetic acid; diflunisal (2′,4′,-difluoro-4-hydroxy-3-biphenylcarboxylic acid; piroxicam (4-hydroxy-2-methyl-N-2-pyridinyl-2H-1,2-benzothiazine-2-carboxamide 1,1-dioxide, meloxicam (4-hydroxy-2-methyl-N-(5-methyl-2-thiazolyl)-2H-1,2-benzothiazine-3-carboxamide 1,1-dioxide); another oxicam; indomethacin (1-(4-chlorobenzoyl)-5-methoxy-2-methyl-H-indole-3-acetic acid); meclofenamate sodium (N-(2,6-dichloro-m-tolyl) anthranilic acid, sodium salt, monohydrate); nabumetone (4-(6-methoxy-2-naphthalenyl)-2-butanon); ketoprofen (2-(3-benzoylphenyl)-propionic acid; tolmetin sodium (sodium 1-methyl-5-(4-methylbenzoyl-1H-pyrrole-2-acetate dihydrate); diclofenac sodium (2-[(2,6-dichlorophenyl)amino]benzeneatic acid, monosodium salt); hydroxychloroquine sulphate (2-{[4-[(7-chloro-4-quinolyl)amino]pentyl]ethylamino}ethanol sulfate (1:1); penicillamine (3-mercapto-D-valine); flurbiprofen ([1,1-biphenyl]-4-acetic acid, 2-fluoro-aphamethyl-, (+−)); etodolac (1-8-diethyl-13,4,9,tetra hydropyrano-[3-4-13]indole-1-acetic acid; mefenamic acid (N-(2,3-xylyl)-anthranilic acid; and diphenhydramine hydrochloride (2-diphenyl methoxy-N,N-di-methylethamine hydrochloride).

According to one embodiment, the analgesic is selected from the group consisting of acetaminophen and dipyrone (4-methylamino-1,5-dimethyl-2-phenyl-3-pyrazolone sodium methanesulfonate).

According to one embodiment, the migraine medication is selected from the group consisting of a triptan; sumatriptan (3-(2-(dimethylamino)ethyl)-N-methyl-1H-indole-5-methanesulfonamide); almotriptan (1-(((3-(2(dimethylamino)ethyl)indol-5-yl)methyl)sulfonyl)pyrrolidine), and amitriptyline (3-(10,11-dihydro-5H-dibenzo (a,d)cyclohepten-5-ylidene)-N,N-dimethyl-1-propanamine).

According to one embodiment, the menopause medication is selected from the group consisting of venlafaxine; paroxetine; a phytoestrogen; and a plant extract. In one embodiment, the plant extract is derived from a plant selected from the group consisting of black cohosh and maca.

According to one embodiment, the sleep disorder medication is selected from the group consisting of diphenhydramine (2-(benzhydryloxy)-N,N-dimethylethylamine); valerian and melatonin (5-methoxy-N-acetyltryptamine).

According to one embodiment, the erectile dysfunction medication is selected from the group consisting of a prostaglandin; a testosterone; yohimbine; pentoxifylline; trazodone; apomorphine; sildenafil; minoxidil; misoprostol; papaverine; nitroglycerin; phentolamine; moxisylyte; linsidomine, and a pyridylguanidine compound.

According to one embodiment, the appetite suppressant is DL-phenylalanine.

According to one embodiment, the DL-phenylalanine is in an amount of about 1% to about 5% by weight of the total weight of the composition. According to one embodiment, the DL-phenylalanine is in an amount of about 5% by weight of the total weight of the composition. According to certain embodiments, the DL-phenylalanine is selected from the group consisting of D-phenylalanine; L-phenylalanine, and a mixture thereof.

According to one embodiment, the macromolecule is selected from the group consisting of vitamin B12; an antibiotic; a peptide; calcitonin; vasopressin and oxytocin. In a particular embodiment, the macromolecule is vitamin B12. In a particular embodiment, the macromolecule has a molecular weight of up to about 5000 Daltons.

According to certain embodiments, the composition of the present invention is formulated in the form of a lozenge, candy, or dissolving tablet.

According to other embodiments, the oral mucosa is selected from the group consisting of the sublingual mucosa; the buccal mucosa; gingival mucosa; palatal mucosa and a combination thereof.

According to another embodiment, the present invention provides a method for enhancing the delivery of a pharmaceutical ingredient across the oral mucosa, the method comprising administering to a subject in need thereof an effective amount of a composition of the invention. In one embodiment, the composition comprises a pharmaceutical ingredient; methylsulfonylmethane; an edible oil; purified water, and lecithin, wherein the methylsulfonylmethane is in an amount sufficient to enhance transmucosal delivery of the pharmaceutical ingredient.

According to a further aspect, the present invention provides a method for suppressing the appetite of a mammal in need thereof, wherein the method comprises transmucosally administering a composition comprising an appetite-suppressing amount of DL-phenylalanine; methylsulfonylmethane; an edible oil; purified water and lecithin, thereby suppressing the appetite of the mammal in need thereof.

According to one embodiment, the transmucosal administration is via the oral mucosa. According to one embodiment, the methylsulfonylmethane is in an amount sufficient to enhance transmucosal delivery of the DL-phenylalanine. According to one embodiment, the mammal is a human. According to one embodiment, the mammal is obese. According to another embodiment, the mammal is morbidly obese.

According to a further aspect, the present invention provides use of methylsulfonylmethane and an appetite-suppressing amount of DL-phenylalanine for the preparation of a medicament for transmucosal oral delivery for suppressing the appetite of a mammal. According to one embodiment, the methylsulfonylmethane is used in an amount sufficient to enhance transmucosal delivery of the DL-phenylalanine.

These and other embodiments of the present invention will become apparent in conjunction with the figures, description and claims that follow.

### BRIEF DESCRIPTION OF THE FIGURE

FIG. 1 shows the effect of methylsulfonylmethane (MSM) on transmucosal-buccal delivery of DL-phenylalanine. DL-phenylalanine concentrations (nmoles/ml) in blood vs. time following buccal administration to humans are presented.

### DETAILED DESCRIPTION OF THE INVENTION

The present invention provides compositions and methods for enhancing transmucosal delivery of numerous pharmaceutical ingredients, including those which are classified as non-steroid anti-inflammatory drugs (NSAIDs), analgesics, migraine medications, menopause medications, sleep disorder medications, erectile dysfunction medications, and appetite suppressants. The composition of the invention is particularly suitable for transmucosal delivery of macromolecules having molecular weight up to about 5000 daltons, and is advantageous over prior art transmucosal delivery formulations which are suitable only for smaller sized molecules, for example up to about 200 daltons. The present invention further provides appetite-suppressing compositions and methods. Accordingly, the disclosed appetite-suppressing compositions can be administered via oral mucosa to effectively suppress, inhibit, reduce or otherwise curtail an appetite in an individual. The appetite-suppressing compositions can be administered to an individual in order to control weight, or to treat obesity or an obesity-related disorder including but not limited to type II diabetes, hypercholesteremia, and metabolic syndrome.

The present invention for the first time discloses that methylsulfonylmethane is an effective enhancer of transmucosal delivery of an active agent. It is particularly suitable for use in delivery of pharmaceutical and nutraceuticals via the oral mucosa.

The effectiveness of MSM is not limited by the type of carrier used and suitable excipients may be selected as is well known in the art to impart to the compositions the desired consistency and other characteristics. The compositions may be liquid semisolid or solid. Conveniently the compositions may be in the form of a candy or confectionary, or in the form of a film suitable for adhesion to the oral mucosa.

According to certain typical embodiments, the composition comprises:

a pharmaceutical or nutraceutical ingredient in an amount that does not exceed about 5%;

methylsulfonylmethane in an amount that does not exceed about 10%;

vegetable oil in an amount that does not exceed about 40%;

purified water in an amount that does not exceed about 30%;

cocoa butter in an amount that does not exceed about 40%;

sucralose in an amount that does not exceed about 0.2%;

a calcium salt in an amount that does not exceed about 20%, and

lecithin in an amount that does not exceed about 20%; wherein the percentages are weight percent based on the total weight of the composition and the total weight of the composition equals 100%.

### DEFINITIONS

As used herein, the term “administering” refers to administration of the composition of the present invention to the mucous membranes of the oral cavity (i.e., oral mucosa). Examples of suitable sites of administration within the oral mucosa include, without limitation, the mucous membranes of the floor of the mouth (sublingual mucosa), the cheeks (buccal mucosa), the gums (gingival mucosa), the roof of the mouth (palatal mucosa), the lining of the lips, and combinations thereof.

As used herein, the term “D,L-phenylalanine (DLPA)”; refers to D-phenylalanine (DPA), L-phenylalanine (LPA) or a mixture thereof.

The term “about” as used herein refers to +/−10% of the indicated amount.

As used herein, the term “macromolecule” refers to a chemical compound having a molecular weight in the range of about 500 to about 5000 daltons (Da).

As used herein, the term “appetite” refers to the instinctive desires necessary to keep up organic life; especially, the desire to eat or an inherent craving to eat.

As used herein, the term “appetite suppressant” refers to a substance, such as a pharmaceutical ingredient, that has activity in suppressing appetite.

As used herein, the term “appetite-suppressing amount” refers to an amount of a substance, such as a pharmaceutical ingredient that substantially suppresses appetite in a subject.

As used herein, the term “amount sufficient to enhance transmucosal delivery” refers to an amount of methylsulfonylmethane which upon formulation with a pharmaceutical ingredient or a nutraceutical agent, and following transmucosal administration to a subject, substantially increases the blood level of the pharmaceutical or nutraceutical ingredient in the subject, and/or the rate of appearance of the pharmaceutical ingredient in the blood, as compared to a formulation of the same pharmaceutical ingredient which lacks methylsulfonylmethane.

As used herein, the term “hunger” refers to an uneasy sensation occasioned by the lack of food.

As used herein, the term “hunger is suppressed” refers to a state in which the sensation of hunger is restrained, inhibited or suppressed, or the intensity of sensation of hunger is subdued or suppressed to varying degrees.

As used herein, the term “obesity” refers to the condition in which the fatty tissue reserve of a mammal, such as a human, is increased to a point at which it is associated with adverse health conditions and/or increased mortality. Obesity may be measured in objective terms, for example, according to a body mass index (BMI) equal to or greater than 30.0, or according to a waist circumference greater than a 102 cm in men and greater than 88 cm in women.

As used herein, the term “safe and effective” refers to a state of being that is, produces, or is capable of producing a predictable, reproducible, desired response of subject to compositions and methods herein, and which are unlikely to produce adverse side effects and/or produces only minimal/minor side effects.

### Administration Via Oral Mucosa

Medicaments taken by mouth and swallowed are absorbed first into the blood perfusing the gastrointestinal tract. The venous drainage from the GI tract is into the blood perfusing the liver. This means that medicaments absorbed from the lumen of gastrointestinal tract are immediately presented to the liver, the major detoxifying organ of the body. In addition to protecting the organism from ingested toxins, the liver also metabolizes medicaments, which are treated in the same way. Blood from the liver then returns to the left side of the heart via the hepatic portal vein and reaches the rest of the systemic circulation. This first pass through the liver may result in the removal of a substantial proportion of an ingested medicament. The first pass effect is more pronounced for some drugs than others.

Certain areas of the alimentary canal have a venous drainage, which does not involve a first pass through the liver. These areas (the mucous membrane of the buccal cavity, under the tongue and the nasopharynx, and also the distal rectum) drain directly into the left side of the heart. The avoidance of the first pass effect is the rationale for the use of buccal, and sublingual formations.

Both sublingual and buccal formulations depend on the efficient transfer of medicament from a hydrophilic vehicle to the mucous membrane of the sublingual or buccal mucosa. Transfer of medicament through the interstices between or through epithelial cells is governed principally by the lipid solubility of the medicament. Where a drug is water insoluble this is a further barrier to absorption from the sublingual area. There are therefore physical and biological limitations on the therapeutic usefulness of lipophilic medicaments, given by mouth and swallowed. The present invention relates to formulations which are particularly suitable for use for administration of pharmaceutical ingredients or medicaments via a mucosal surface such as, for example, the sublingual mucosa or the buccal mucosa.

According to the present invention, methylsulfonylmethane improves the oral mucosal absorption of a pharmaceutical ingredient such as DL-phenylalanine.

### Pharmaceutical Ingredients

NSAIDs which may be incorporated into the composition of the invention include ibuprofen (2-(isobutylphenyl)-propionic acid); methotrexate (N-[4-(2,4 diamino 6-pteridinyl-methyl]methylamino]benzoyl)-L-glutamic acid); aspirin (acetylsalicylic acid); salicylic acid; diphenhydramine (2-(diphenylmethoxy)-N,N-dimethylethylamine hydrochloride); naproxen (2-naphthaleneacetic acid, 6-methoxy-9-methyl-, sodium salt, (−)); phenylbutazone (4-butyl-1,2-diphenyl-3,5-pyrazolidinedione); sulindac-(2)-5-fluoro-2-methyl-1-[[p-(methylsulfinyl)phenyl]methylene-]-1-H-indene-3-acetic acid; diflunisal (2′,4′,-difluoro-4-hydroxy-3-biphenylcarboxylic acid; piroxicam (4-hydroxy-2-methyl-N-2-pyridinyl-2H-1,2-benzothiazine-2-carboxamide 1,1-dioxide, meloxicam (4-hydroxy-2-methyl-N-(5-methyl-2-thiazolyl)-2H-1,2-benzothiazine-3-carboxamide 1,1-dioxide); an oxicam; indomethacin (1-(4-chlorobenzoyl)-5-methoxy-2-methyl-H-indole-3-acetic acid); meclofenamate sodium (N-(2,6-dichloro-m-tolyl) anthranilic acid, sodium salt, monohydrate); nabumetone (4-(6-methoxy-2-naphthalenyl)-2-butanon); ketoprofen (2-(3-benzoylphenyl)-propionic acid; tolmetin sodium (sodium 1-methyl-5-(4-methylbenzoyl-1H-pyrrole-2-acetate dihydrate); diclofenac sodium (2-[(2,6-dichlorophenyl)amino]benzeneatic acid, monosodium salt); hydroxychloroquine sulphate (2-{[4-[(7-chloro-4-quinolyl)amino]pentyl]ethylamino}ethanol sulfate (1:1); penicillamine (3-mercapto-D-valine); flurbiprofen ([1,1-biphenyl]-4-acetic acid, 2-fluoro-aphamethyl-, (+−)); etodolac (1-8-diethyl-13,4,9,tetra hydropyrano-[3-4-13]indole-1-acetic acid; mefenamic acid (N-(2,3-xylyl)anthranilic acid; and diphenhydramine hydrochloride (2-diphenyl methoxy-N,N-di-methylethamine hydrochloride).

Analgesic compounds which may be incorporated into the composition of the invention include acetaminophen and dipyrone (4-methylamino-1,5-dimethyl-2-phenyl-3-pyrazolone sodium methanesulfonate).

Migraine medications which may be incorporated into the composition of the invention include triptans, such as sumatriptan (3-(2-(Dimethylamino)ethyl)-N-methyl-1H-indole-5-methanesulfonamide) and almotriptan 1-(((3-(2(dimethylamino)ethyl)indol-5-yl)methyl)sulfonyl)pyrrolidine and amitriptyline 3-(10,11-dihydro-5H-dibenzo (a,d)cyclohepten-5-ylidene)-N,N-dimethyl-1-propanamine.

Erectile dysfunction medications which may be incorporated into the composition of the invention include a steroid hormone such as testosterone, a peptide hormone, an amine hormone, and a hormone-like eicosanoid such as a prostaglandin, a leukotriene, and a thromboxane.

The term “prostaglandin” refers to a family of compounds originally discovered in seminal fluid and found to cause vasodilation, and contraction or relaxation of uterine smooth muscle. The prostaglandins, leukotrienes, and related compounds are called eicosanoids because they are synthesized by microsomal enzymes from 20-carbon essential fatty acids, e.g., arachidonic acid (Hardman, J. 1996, in Goodman and Gilmans's: The Pharmacological Basis of Therapeutics, Ch. 26, 9th ed., McGraw Hill).

Other pharmaceutical ingredients which may be used as erectile dysfunction medications include testosterone, yohimbine, pentoxifylline, trazodone, apomorphine, phentolamine, tadalafil, sildenafil and other pyrozolopyrimidone derivatives. Other agents include minoxidil, misoprostol, papaverine, nitroglycerin, phentolamine, moxisylyte, linsidomine, linear or cyclic peptides, pyridylguanidine compounds, and renin-angiotensin system inhibitors. These agents may be incorporated into the subject transmucosal composition at an effective dose to correct erectile dysfunction.

Menopause medications which may be incorporated into the composition of the invention include phytoestrogens, venlafaxine, paroxentine and plant extracts, such as those derived from black cohosh and maca.

Sleep disorder medications which may be incorporated into the composition of the invention include diphenhydramine (2-(benzhydryloxy)-N,N-dimethylethylamine), valerian and melatonin (5-methoxy-N-acetyltryptamine).

An appetite suppressant which may be incorporated into the composition of the invention is DL-phenylalanine. The DL-phenylalanine may be D-phenylalanine, L-phenylalanine or a mixture thereof.

Macromolecules which may be incorporated into the composition of the invention include vitamin B12; antibiotics, peptides, polypeptides, calcitonin, vasopressin and oxytocin. In a particular embodiment, the macromolecule is vitamin B12.

### Pharmaceutical Compositions

The present invention provides a composition for enhanced delivery across the oral mucosa, the composition comprising a pharmaceutical ingredient; methylsulfonylmethane; an edible oil; purified water, and lecithin.

According to one embodiment, the edible oil is a vegetable oil. Exemplary vegetable oils include, but are not limited to cottonseed oil, peanut oil, poppyseed oil, safflower oil, sesame oil, soybean oil, corn oil, olive oil, canola oil and combinations thereof.

The composition may further comprise at least one excipient, such as a sweetening agent, a flavoring agent, a protecting agent, a plasticizer, a wax, an elastomeric solvent, a filler material, a preservative, a lubricating agent, a wetting agent, an emulsifying agent, solubilizing agent, a suspending agent, a coloring agent, a disintegrating agent, or combinations thereof. It is to be understood that some excipients may fall within more than one class of the aforementioned excipients.

According to some embodiments, the composition comprises at least one excipient selected from the group consisting of cocoa butter, sucralose, a calcium salt and combinations thereof. One example of a calcium salt is calcium carbonate.

According to one embodiment, the composition comprises a pharmaceutical ingredient in an amount of about 1% to 5% of the composition, wherein the percentages are weight percent based on the total weight of the composition and the total weight of the composition equals 100%. In one embodiment, the pharmaceutical ingredient is in an amount of about 5% of the composition. In certain embodiments, the pharmaceutical ingredient is in an amount of about 2% to 4% of the composition.

According to one embodiment, the methylsulfonylmethane is in an amount of about 1% to 10% of the composition, wherein the percentages are weight percent based on the total weight of the composition and the total weight of the composition equals 100%. In certain embodiments, the methylsulfonylmethane is present in an amount of about 2% to 5% of the composition.

According to another embodiment, the vegetable oil is in an amount of about 20% to 40% of the composition. In certain embodiments, the vegetable oil is in an amount of about 25% to 35% of the composition.

According to a further embodiment, the purified water is in an amount of about 10% to 30% of the composition. In certain embodiments, the purified water is in an amount of about 15% to 25% of the composition.

According to still another embodiment, the cocoa butter is in an amount of about 20% to 40% of the composition. In certain embodiments, the cocoa butter is in an amount of about 25% to 35% of the composition.

According to still a further embodiment, the sucralose is in an amount of about 0.05% to 0.2% of the composition. In certain embodiments, the sucralose is in an amount of about 0.07% to 0.15% of the composition.

According to yet another embodiment, the calcium salt is in an amount of about 1% to 20% of the composition. In certain embodiments, the calcium salt is in an amount of about 5% to 15% of the composition. In some embodiments, the calcium salt is calcium carbonate.

According to yet a further embodiment, lecithin is in an amount of about 1% to 20% of the composition. In certain embodiments, the lecithin is in an amount of about 5% to 15% of the composition.

The present invention relates to pharmaceutical formulations for use in the administration of a pharmaceutical ingredient such as DL-phenylalanine via mucosal surfaces.

According to one embodiment, the composition of the present invention is directed particularly at weight loss, and comprises the appetite suppressant DL-phenylalanine as the pharmaceutical ingredient.

### Production of the Composition of the Present Invention

Exemplary solid forms for oral mucosal (e.g. sublingual and buccal) administration include films, dispersible fluid or semisolid compositions as well as solid forms. Solid dosage forms intended for dispersion on or at the oral mucosa may conveniently be designed to take the form of a hard candy or a chewable confectionary form. Other forms may include tablets, pills, capsules, powders and granular substances, useful for dispersion in the oral cavity. These solid compositions can include pharmaceutically acceptable inert ingredients such as diluents (e.g. calcium carbonate, sodium chloride, lactose, calcium phosphate, sodium phosphate, and the like); granulating and disintegrating agents (e.g. potato starch, alginic acid and the like); binding agents (e.g. starch, gelatin, acacia and the like); lubricating agents (e.g. magnesium stearate, stearic acid, talc and the like). Other inert ingredients that can be used in the invention include colorants, flavoring agents, plasticizers, humectants and the like. The solid compositions provided in accordance with the present invention can be uncoated or they can be coated by methods and using materials known in the art.

The composition of the present invention can be produced by various methods. The production techniques available for the conventional tablets, chewing gum species, or candies, for instance, can be used in the production of the composition of the present invention.

In a typical process for the production of the composition, the water, methylsulfonylmethane, DL-phenylalanine, sucralose, lecithin, cocoa butter, vegetable oil and calcium carbonate are mixed, the mixture is kneaded uniformly, compressed and extended while it is warm, and then, after cooling, the resulting sheet is cut.

Liposomes can be incorporated into the tablet by adding lecithin and vegetable oil in solution to form liposomes as the pharmaceutical solution, and then gently vortexing to prevent damage to the liposomes before introduction into molds where gelling occurs.

In an alternative process, the above-mentioned mixture is pulverized as it is without addition of water and then compression-molded. In a third process, the above-mentioned mixture is uniformly dispersed or dissolved in water and thus molded by the wet method, followed by drying. The steps in these production processes may be combined in an adequate manner other than the manners mentioned above. In producing the composition according to the present invention, the components are used in the amounts prescribed above.

In the step of molding or shape adjustment as mentioned above, compositions having a desired shape, such as the plate, belt-, disk-, pillar-, cylinder- or spindle-like form, can be obtained by adjusting the mold for compression molding to an appropriate shape or by pouring into a mold or extending the molding compound followed by cutting the previously formed moldings into pieces having an appropriate shape. Such a production method comprises only simple and easy operations and steps and accordingly is an advantageous production method usable on a commercial scale, by which compositions in various shapes suited for the purpose in each individual case can be produced depending on the procedure followed. When a step of dissolution or dispersion is included, liquid or low-melting drugs, which are hard to incorporate into solid preparations, can be incorporated into the composition.

In addition to the above components, there may also be incorporated other additives selected from among the various pharmaceutically acceptable additives available to those skilled in the art for the purpose of assisting in the development of characteristics of the composition of the present invention, by improving the processability, moldability and quality of the preparation, and by enhancing the dispersability and stability of the composition. Such additives are other than those mentioned as the essential components and include the following substances.

Flavors (saccharin sodium, glycyrrhizin, malt syrup, citric acid, tartaric acid, menthol, lemon oil, citrus flavor, common salt, etc.);

Stabilizers/preservatives (parahydroxybenzoic acid alkyl esters, antioxidants, antifungal agents, etc.);

Colors (water-soluble tar colors, natural colors, titanium oxide, etc.);

Excipients/disintegration adjusting agents (magnesium silicate, light silicic acid anhydride, synthetic aluminum silicate, precipitated calcium carbonate, magnesium aluminum metasilicate, calcium hydrogen phosphate, etc.);

Water-soluble polymers other than water-soluble proteins (natural polymers, synthetic polymers, etc.) and

Stearic acid and its salts, talc, palmitic acid, and other substances known as emulsifiers, dispersants, binders, thickeners, etc.

According to certain preferred embodiments the composition of the present invention is preferably formulated in the form of a lozenge, candy, or dissolving tablet (e.g., slow-dissolving tablet or quick-dissolving tablet) for transmucosal-oral administration.

In certain embodiments, the tablet is a dissolving tablet such as a slow-dissolving or quick-dissolving tablet that is dissolved by a subject's saliva, without the need for chewing. For example, a dissolving tablet placed on the subject's tongue can be used for buccal delivery of the therapeutic agent. Alternatively, a dissolving tablet placed underneath the subject's tongue can be used for sublingual delivery of the therapeutic agent. This type of dosage form may be particularly desirable for pediatric and geriatric patients, since small children and aged individuals often have difficulty chewing certain items. Typically, the dissolving tablet is formulated to dissolve within about 30 seconds to about 5 minutes, preferably within about 1 to about 3 minutes following administration. One skilled in the art will understand that quick-dissolving tablets dissolve faster than slow-dissolving tablets, which are typically dissolved gradually rather than rapidly by a subject's saliva.

In certain other embodiments, the tablet is a chewable tablet that is chewed by a subject and formulated to dissolve either rapidly or gradually. For example, a chewable tablet placed on the subject's tongue can be used for buccal delivery of the therapeutic agent. During chewing, the chewable tablet can be moved around within the mouth and can sometimes be parked between the gums and the cheeks or underneath the tongue. As a result, at least a portion of the therapeutic agent contained within a chewable tablet may also be delivered sublingually (i.e., across the sublingual mucosa). Typically, the chewable tablet is formulated to dissolve within about 30 seconds to about 5 minutes, preferably within about 1 to about 3 minutes following administration.

The present oral dosage form can be used to deliver any active or therapeutic agent where absorption across the oral mucosa is desired. While according to an exemplary embodiment, the present invention will hereinafter be discussed with reference to administration of the appetite suppressant DL-phenylalanine, it should be understood that other actives may be adjunctively or alternatively employed.

Non-limiting examples include non-steroid anti-inflammatory drugs, analgesics, migraine medications, menopause medications, sleep disorder medications, erectile dysfunction medications, appetite suppressants, cough/cold/throat agents, vitamins, zinc, menthol, eucalyptus, hexyl resorcinol, caffeine, tooth whitening agents, anti-plaque agents, breath freshening agents, demulcents and the like.

The composition is orally dissolvable and may be in any form which is typically sucked, licked, and/or chewed and eaten, such as tablets, lozenges, sticks, canes, pops, and the like. Tablets and lozenges are preferred forms. Tablets and lozenges of the present invention are oral dosage forms intended to be held in the mouth, and are typically sucked. For example, they may be held in the buccal cavity or sublingually. The tablets or lozenges may be in various shapes, including flat, circular, octagonal and biconvex. According to a preferred embodiment, the tablet is a chocolate flavored tablet.

According to other embodiments the oral dosage form may be a film designed to adhere to the oral mucosa. For example U.S. Pat. No. 4,713,243 to Schiraldi et al. discloses a thin film capable of adhering to a wet mucous surface which comprises a bioadhesive layer consisting of hydroxypropyl cellulose, polyethylene oxide, a plasticizer, a medicament, and optionally a water-insoluble polymer. The film disclosed in U.S. Pat. No. 4,713,243 is useful for controlled release of a medicament such as anesthetics, anti-inflammatories, antihistamines, antibiotics, and antibacterials.

U.S. Pat. Nos. 5,948,430, 6,177,096, 6,284,264, 6,592,887, and 6,709,671 to Zerbe et al. disclose mucoadhesive films capable of rapidly dissolving and adhering to the oral cavity comprising a water-soluble polymer and a pharmaceutically or cosmetically active ingredient and methods of use thereof.

### Methods for Appetite Suppression

The invention further provides a method for suppressing the appetite of a mammal in need thereof, wherein the method comprises transmucosally administrating a composition comprising an appetite-suppressing amount of DL-phenylalanine, methylsulfonylmethane, an edible oil, purified water and lecithin, thereby suppressing the appetite of the mammal in need thereof.

The composition is preferably formulated as an orally administratable dosage form, such as a tablet, which thus enables transmucosal administration via the oral mucosa. The methylsulfonylmethane is present in the composition in an amount sufficient to enhance transmucosal delivery of the DL-phenylalanine. That is, the amount of methylsulfonylmethane in the composition is sufficient to substantially increase the blood level of DL-phenylalanine in a subject following transmucosal administration thereto, and/or the rate of appearance of the DL-phenylalanine in the blood, as compared to a formulation of the same pharmaceutical ingredient which lacks methylsulfonylmethane. As exemplified in Example 2 herein, a transmucosal oral composition comprising DL-phenylalanine and methylsulfonylmethane according to the invention provides an increased Cₘₐₓ and a decreased Tₘₐₓ following administration to human subjects, in comparison to a similar composition lacking methylsulfonylmethane.

Advantageously, transmucosal delivery of the appetite suppressant DL-phenylalanine offers a rapid response to hunger cravings and appetite urges which are apparently part of the cycle of overeating in obese and morbidly obese subjects. Furthermore, the provision of an appetite suppressant in a transmucosal-oral dosage form addresses the need for oral gratification which is prevalent in many obese subjects.

Accordingly, the composition and method of the invention, employing the appetite suppressant DL-phenylalanine and the transmucosal delivery enhancer methylsulfonylmethane, can be used in programs of weight loss and weight control in overweight subjects, including those who are classified as obese and morbidly obese.

According to one embodiment, the mammal is a human. According to one embodiment, the mammal is obese. According to another embodiment, the mammal is morbidly obese.

Classification of a subject as obese or morbidly obese is within the ability of one of average skill in the art, and may include use of objective parameters such as body mass index (BMI); waist circumference measurement and/or body fat measurements.

BMI is calculated according to the formula BMI=kg/m², where kg is the weight of the subject in kilograms and m is the height of the subject in meters.

The following guidelines (according to World Health Organization Technical report series 894, “Obesity: preventing and managing the global epidemic.”, Geneva: World Health Organization, 2000) may be used to determine obesity:

| BMI | Definition |
|---|---|
| less than 18.5 | Underweight |
| 18.5-24.9 | Normal weight |
| 25.0-29.9 | Overweight |
| 30.0-39.9 | Obese |
| 40.0 or higher | Morbidly obese |
| 35.0 or higher in the | Morbidly obese |
| presence of at least | |
| one other significant | |
| comorbidity | |

In a clinical setting, physicians take into account race, ethnicity, lean mass (muscularity), age, sex, and other factors which can affect the interpretation of BMI. BMI overestimates body fat in persons who are very muscular, and it can underestimate body fat in persons who have lost body mass (e.g. many elderly).

BMI does not take into account differing ratios of adipose to lean tissue, nor does it distinguish between differing forms of adiposity, some of which may correlate more closely with cardiovascular risk. Increasing understanding of the biology of different forms of adipose tissue has shown that visceral fat or central obesity (male-type or apple-type obesity) has a much stronger correlation, particularly with cardiovascular disease, than the BMI alone.

The absolute waist circumference (>102 cm in men and >88 cm in women) or waist-hip ratio (>0.9 for men and >0.85 for women) are both used as measures of central obesity.

An alternative means to determine obesity is to assess percent body fat. It is generally agreed that men with more than 25% body fat and women with more than 30% body fat are obese. The most accepted method to measure body fat precisely has been to weigh a person underwater, a procedure limited to laboratories with special equipment. Two simpler methods for measuring body fat are the skinfold test, in which a pinch of skin is precisely measured to determine the thickness of the subcutaneous fat layer; or bioelectrical impedance analysis. Other measurements of body fat include computed tomography (CT/CAT scan), magnetic resonance imaging (MRI/NMR), and dual energy X-ray absorptiometry (DXA).

According to a further aspect, the present invention provides use of methylsulfonylmethane and an appetite-suppressing amount of DL-phenylalanine for the preparation of a medicament for delivery across the oral mucosa for suppressing the appetite of a mammal.

Having now generally described the invention, the same will be more readily understood through reference to the following examples, which are provided by way of illustration and are not intended to be limiting of the present invention.

### EXAMPLES

### Example 1

### Preparation of a Composition Comprising DL-Phenylalanine

A soft buccal dosage formulation in the form of chocolate tablets weighing 3 grams each was prepared.

Each tablet contains:

DL-Phenylalanine 150 mg

Methylsulfonylmethane (MSM) 100 mg

Vegetable oil 30%

Cocoa butter 30%

Sucralose 0.1%

Calcium carbonate 10%

Lecithin 10% and

Purified water to 100%

The method for preparation of the composition comprising the steps of:
Step 1: 20 ml of de-ionized water are poured to a homogenizing system and heated to a temperature of 50° C.
Step 2: MSM is added to the water and homogenized thoroughly.
Step 3: Phenylalanine is added to the water and homogenized thoroughly.
Step 4: Sucralose is added to the water and homogenized thoroughly.
Step 5: The ingredients are thoroughly mixed for 30 minutes to produce solution A.
Step 6: Lecithin is added to solution A and mixed thoroughly for 60 minutes.
Step 7: Cocoa butter is added to vegetable oil and mixed thoroughly for 30 minutes.
Step 8: Cocoa butter and vegetable oil mixture is added to the solution and mixed thoroughly for 30 minutes.
Step 9: Calcium carbonate is added to the solution and mixed thoroughly for 30 minutes.
Step 10: The solution is poured to sterilize containers and is left for 24 hours.

### Example 2

### Comparison in Bioavailability of Preparations Containing DL-Phenylalanine in the Presence or Absence of Methylsulfonylmethane (MSM)

Three healthy adult male subjects were given tablets prepared by the method described in EXAMPLE 1, between the gingiva and the cheek in the oral cavity at the dose of 450 mg DL-phenylalanine/subject. (A) a preparation without MSM at retaining time (in oral cavity) of 60 sec; (B) a preparation with MSM at retaining time (in oral cavity) of 30 sec; and (C) a preparation with MSM at retaining time (in oral cavity) of 60 sec. The subjects were fasted from 12 hours before the administration to the completion of the test.

Blood samples (3 ml) were taken before the administration and at 20, 40, 60, and 80 min after the administration of the preparations. The determination of DL-phenylalanine in blood was performed by the following analytical method.

After blood separation, plasma was stored at ±20° C., until the chemical analysis was performed. The plasmas with added internal standard, were deproteinized with 35% sulphosalicyclic acid, centrifuged and filtered through a 0.2 u filter. The filtrate was used for the analysis. The instrument used was Biochrom 20 plus dedicated amino acid analyzer (Pharmacia Biotech Ltd. Cambridge, England). Separation of the amino acids is achieved on an ion exchange column. The resin used is a sulphonated polystiren cross-liked with divinylbenzene.

Final separation is achieved by alteration in the column temperature changing the buffer PH, and the alteration in the time that the buffer is pumped. Once separation has been achieved the eluted amino acid is reached with ninhydrin and the color is read photometrically at 570 nm and 440 nm. The digital signal corresponding to the color is read by the computer, integrated, and compared to known standards and the concentration of each is then measured. The data handling software that is used to collect and process the data is EZCrome ELITE™ CLIENT/SERVER Chromatography software (Scientific Software Inc, Pleasanton, Calif.).

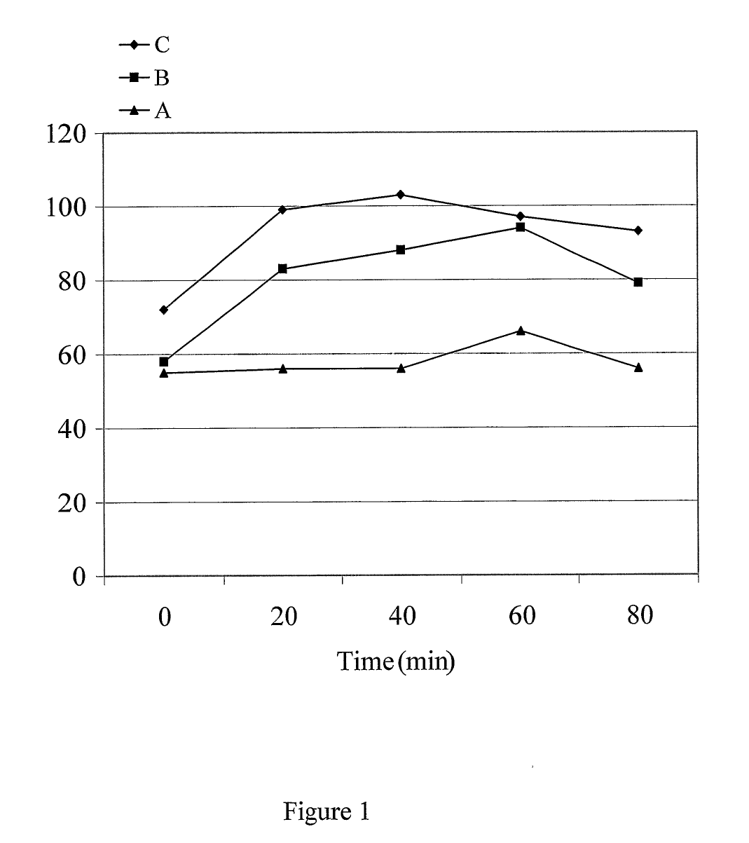

The DL-phenylalanine levels in blood after buccal administration of exemplary preparations are presented in FIG. 1.

As shown in FIG. 1, the maximal blood DL-phenylalanine level (Cₘₐₓ) in the presence of MSM was about 103 nmole/ml, and the time required for the maximal blood level to be attained (Tₘₐₓ) was 40 min (subject C), whereas the maximal blood DL-phenylalanine level (Cₘₐₓ) in the absence of MSM was about 66 nmole/ml and the time required for the maximal blood level to be attained (Tₘₐₓ) was 60 min (subject A). Thus, the presence of MSM in the preparation increases the buccal absorption of the DL-phenylalanine.

### Example 3

### The Effect of the Composition of the Invention on Weight Loss

A 90-day, double-blind retrospective study is conducted to investigate the effect of the composition described in EXAMPLE 2 on weight loss. The composition was compared to the effects of a placebo tablet.

A test panel is selected of 40 individuals, who are an average of 60 pounds overweight. The individuals are divided into two groups. Group I consists of 20 members and is receiving 6 placebo tablets daily. Group II consists of 20 members and is receiving 6 tablets of the composition daily. Both groups are receiving 3 daily doses of 2 tablets each, taken 30 minutes before meals. All patients are provided with dietary calendars and are asked to record their daily intake of foods and liquids. Consultations are held weekly between patients and a physician or other medical professional. Patients are encouraged to discuss their weight loss program, including successes, difficulties, or topics of concern to them. During these weekly consultations, patients are weighed and measurements are taken to record blood pressure, pulse rate and respiration. Patients are asked to report their energy levels, exercise activity, cravings for sugar and carbohydrates, any changes in appetite, and any possible side effects of the course of treatment.

Weight loss is measured and recorded in three ways: (1) Total weight loss from the patient's initial weight; (2) reduction in excess weight; and (3) percentage of the excess weight that was lost. The data are obtained in the double-blind, retrospective clinical study in order to test whether the composition of the invention is effective in reducing feelings of hunger and therefore in reducing overeating in overweight individuals.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It should be understood that the detailed description and specific examples, while indicating preferred embodiments of the invention, are given by way of illustration only, since various changes and modifications within the spirit and scope of the invention will become apparent to those skilled in the art from this detailed description.

1-56. (canceled)

57. A pharmaceutical or nutraceutical composition for enhanced delivery through the oral mucosa, the composition comprising a pharmaceutical or nutraceutical ingredient and methylsulfonylmethane in a carrier suitable for application to the oral mucosa, wherein the carrier comprises an edible oil, water and lecithin.

58. The composition of claim 57, wherein the edible oil is a vegetable oil.

59. The composition of claim 58, wherein the vegetable oil is selected from the group consisting of cottonseed oil, peanut oil, poppyseed oil, safflower oil, sesame oil, soybean oil, corn oil, olive oil, canola oil, and combinations thereof.

60. The composition of claim 57, wherein the composition further comprises at least one excipient selected from the group consisting of a sweetening agent, a flavoring agent, a protecting agent, an antioxidant, a plasticizer, a wax, an elastomeric solvent, a filler, a preservative, a lubricating agent, a wetting agent, an emulsifying agent, solubilizing agent, a suspending agent, a coloring agent, a disintegrating agent and combinations thereof.

61. The composition of claim 60, wherein the composition comprises at least one excipient selected from the group consisting of cocoa butter, sucralose, a calcium salt and combinations thereof.

62. The composition of claim 57, wherein the composition comprises:
a pharmaceutical ingredient in an amount of about 0.1 to 15%;
methylsulfonylmethane in an amount of about 0.5-30%;
a vegetable oil in an amount of about 5-40%;
purified water in an amount of about 5-30%;
cocoa butter in an amount of about 5-40%;
a calcium salt in an amount of about 1-20% and
lecithin in an amount that does not exceed about 1-20%; wherein the percentages are weight percent based on the total weight of the composition and the total weight of the composition equals 100%.

63. The composition of claim 62, wherein the pharmaceutical ingredient is in an amount of about 1% to 5%;
the methylsulfonylmethane is in an amount of about 1% to 10%;
the vegetable oil is in an amount of about 20% to 40%;
the purified water is in an amount of about 10% to 30%;
the cocoa butter is in an amount of about 20% to 40%;
the calcium salt is in an amount of about 1% to 20% and
the lecithin is in an amount of about 1% to 20%.

64. The composition of claim 62, wherein the pharmaceutical ingredient is in an amount of about 2% to 4%;
the methylsulfonylmethane is in an amount of about 2% to 5%;
the vegetable oil is in an amount of about 25% to 35%;
the purified water is in an amount of about 15% to 25%;
the cocoa butter is in an amount of about 25% to 35%;
the calcium salt is in an amount of about 5% to 15% and
the lecithin is in an amount of about 5% to 15%.

65. The composition of claim 57, wherein the pharmaceutical ingredient is selected from the group consisting of a non-steroid anti-inflammatory drug (NSAID); an analgesic; a migraine medication; a menopause medication; a sleep disorder medication; an erectile dysfunction medication, an appetite suppressant and a macromolecule.

66. The composition of claim 65, wherein the NSAID is selected from the group consisting of ibuprofen (2-(isobutylphenyl)-propionic acid); methotrexate (N-[4-(2,4 diamino 6-pteridinyl-methyl]methylamino]benzoyl)-L-glutamic acid); aspirin (acetylsalicylic acid); salicylic acid; diphenhydramine (2-(diphenylmethoxy)-N,N-dimethylethylamine hydrochloride); naproxen (2-naphthaleneacetic acid, 6-methoxy-9-methyl-, sodium salt, (−)); phenylbutazone (4-butyl-1,2-diphenyl-3,5-pyrazolidinedione); sulindac-(2)-5-fluoro-2-methyl-1-[[p-(methylsulfinyl)phenyl]methylene-]-1-H-indene-3-acetic acid; diflunisal (2′,4′,-difluoro-4-hydroxy-3-biphenylcarboxylic acid; piroxicam (4-hydroxy-2-methyl-N-2-pyridinyl-2H-1,2-benzothiazine-2-carboxamide 1,1-dioxide, meloxicam (4-hydroxy-2-methyl-N-(5-methyl-2-thiazolyl)-2H-1,2-benzothiazine-3-carboxamide 1,1-dioxide); an oxicam; indomethacin (1-(4-chlorobenzoyl)-5-methoxy-2-methyl-H-indole-3-acetic acid); meclofenamate sodium (N-(2,6-dichloro-m-tolyl) anthranilic acid, sodium salt, monohydrate); nabumetone (4-(6-methoxy-2-naphthalenyl)-2-butanon); ketoprofen (2-(3-benzoylphenyl)-propionic acid; tolmetin sodium (sodium 1-methyl-5-(4-methylbenzoyl-1H-pyrrole-2-acetate dihydrate); diclofenac sodium (2-[(2,6-dichlorophenyl)amino]benzeneatic acid, monosodium salt); hydroxychloroquine sulphate (2-{[4-[(7-chloro-4-quinolyl)amino]pentyl]ethylamino}ethanol sulfate (1:1); penicillamine (3-mercapto-D-valine); flurbiprofen ([1,1-biphenyl]-4-acetic acid, 2-fluoro-aphamethyl-, (+−)); etodolac (1-8-diethyl-13,4,9,tetra hydropyrano-[3-4-13]indole-1-acetic acid; mefenamic acid (N-(2,3-xylyl)anthranilic acid; and diphenhydramine hydrochloride (2-diphenyl methoxy-N,N-di-methylethamine hydrochloride).

67. The composition of claim 65, wherein the analgesic is selected from the group consisting of acetaminophen and dipyrone (4-methylamino-1,5-dimethyl-2-phenyl-3-pyrazolone sodium methanesulfonate).

68. The composition of claim 65, wherein the migraine medication is selected from the group consisting of a triptan; sumatriptan (3-(2-(dimethylamino)ethyl)-N-methyl-1H-indole-5-methanesulfonamide); almotriptan (1-(((3-(2(dimethylamino)ethyl)indol-5-yl)methyl)sulfonyl)pyrrolidine) and amitriptyline (3-(10,11-dihydro-5H-dibenzo (a,d)cyclohepten-5-ylidene)-N,N-dimethyl-1-propanamine).

69. The composition of claim 65, wherein the menopause medication is selected from the group consisting of venlafaxine, paroxetine, a phytoestrogen and a plant extract.

70. The composition of claim 69, wherein the plant extract is derived from a plant selected from the group consisting of black cohosh and maca.

71. The composition of claim 65, wherein the sleep disorder medication is selected from the group consisting of diphenhydramine (2-(benzhydryloxy)-N,N-dimethylethylamine); valerian, and melatonin (5-methoxy-N-acetyltryptamine).

72. The composition of claim 65, wherein the erectile dysfunction medication is selected from the group consisting of a prostaglandin; a testosterone; yohimbine; pentoxifylline; trazodone; apomorphine; sildenafil; tadalafil; minoxidil; misoprostol; papaverine; nitroglycerin; phentolamine; moxisylyte; linsidomine, and a pyridylguanidine compound.

73. The composition of claim 65, wherein the macromolecule is selected from the group consisting of vitamin B12; an antibiotic; a peptide; calcitonin; vasopressin and oxytocin.

74. The composition of claim 65, wherein the appetite suppressant is DL-phenylalanine.

75. The composition of claim 74, wherein the DL-phenylalanine is in an amount of about 1% to 5% by weight of the total weight of the composition.

76. The composition of claim 57, wherein the composition is a sublingual or buccal dosage form selected from the group consisting of a lozenge, a chewing gum, a chewable tablet, and a dissolving tablet.

77. A method for enhancing the delivery of a pharmaceutical ingredient across the oral mucosa, the method comprising administering to a subject in need thereof a composition comprising a pharmaceutical ingredient and methylsulfonylmethane in a carrier suitable for application to the oral mucosa; wherein the methylsulfonylmethane is in an amount sufficient to enhance transmucosal delivery of the pharmaceutical ingredient.

78. The method of claim 77, wherein the carrier comprises an edible oil, purified water, and lecithin.

79. A method for suppressing the appetite of a mammal in need thereof, wherein the method comprises administering transmucosally, through the oral mucosa, a composition comprising an appetite-suppressing amount of DL-phenylalanine; in a carrier comprising methylsulfonylmethane; an edible oil; purified water and lecithin, thereby suppressing the appetite of the mammal in need thereof.

80. The method of claim 79, wherein the edible oil is a vegetable oil.

81. The method of claim 79, wherein the oral mucosa is selected from the group consisting of the sublingual mucosa, the buccal mucosa, gingival mucosa, palatal mucosa and a combination thereof.
